(19) United States
(12) Patent Application Publication
(10) Pub. No.: US 2014/0118693 A1
(43) Pub. Date: May 1, 2014

(54) OPHTHALMOLOGIC APPARATUS, CONTROL METHOD THEREOF, AND PROGRAM

(71) Applicant: CANON KABUSHIKI KAISHA, Tokyo (JP)

(72) Inventor: Ryutaro Matsuoka, Tokyo (JP)

(21) Appl. No.: 14/063,256

(22) Filed: Oct. 25, 2013

(30) Foreign Application Priority Data
Oct. 26, 2012 (JP) 2012-236458

(51) Int. Cl. A61B 3/12

(52) U.S. Cl. CPC A61B 3/12
USPC 351/206; 351/246

(57) ABSTRACT

An ophthalmologic apparatus includes an illumination projection unit configured to project an illumination light onto a fundus of a subject's eye, an index projection unit configured to project an index onto the subject's eye, an image capturing unit configured to capture a fundus image including an index image based on light returning from the subject's eye, a focusing unit configured to focus the light returning from the subject's eye onto the photographic unit, a detection unit configured to detect a position of the index image in the fundus image, a determination unit configured to determine the area in the fundus image which does not include the index image based on results detected by the detection unit, and a control unit configured to control the focusing unit based on contrast of the area determined by the determination unit.

### BACKGROUND

1. Field

Aspects of the present invention generally relate to an ophthalmologic apparatus, a control method thereof, and a program which are particularly suitable for a fundus camera equipped with an automatic focusing mechanism.

2. Related Art

In a conventional fundus camera, two main methods have been known as a method for focusing light on the fundus of a subject's eye. In one method a focus index is projected onto the subject's eye fundus and focusing is performed using a focus index image reflected from the fundus. In the other method focusing is performed by detecting the contrast of the subject's eye fundus.

Japanese Patent Application Laid-Open No. 5-95907 using the former method discusses a fundus camera in which the focus index is projected onto the fundus of a subject's eye, and a photographic optical system captures a focus index image to detect a focus state. Japanese Patent Application Laid-Open No. 5-95907 further discusses a technique for shielding and attenuating a light source for detecting a fixation target and working distance which are not required at the time of automatic focusing to improve the accuracy of the automatic focusing.

Japanese Patent Application Laid-Open No. 2011-50532 using the latter method discusses a fundus camera in which a specific site on the fundus is predicted by detecting the line of sight, and left and right eyes, to detect a focus detection area and contrast inside the area is detected to perform automatic focusing.

However, in Japanese Patent Application Laid-Open No. 5-95907 which uses the former method, if the light source for index for detecting the fixation target and index for detecting a working distance is attenuated, the subject loses the fixation target which deviates the line of sight or the subject is liable to become unaware of alignment displacement. In other words, the focusing is performed with displaced alignment.

According to Japanese Patent Application Laid-Open No. 2011-50532 using the latter method, when the automatic focusing is performed by detecting the contrast of the fundus image, the specific site is predicted from line of sight and left and right eyes to determine the focus detection area, the images other than the fundus images in the area may be captured. The images other than the fundus images refer to images of index for detecting the fixation target and the working distance, or focus index, for example. Thus, the images other than the fundus images captured in the focus detection area make the accuracy of automatic focusing lower than the case where only the contrast of the fundus image is detected. In other words, contrast evaluation is not correctly performed and focusing is completed in an out-of-focus state.

### SUMMARY

The embodiments disclosed herein are directed to an ophthalmologic apparatus, a control method thereof, and a program which are capable of accurately performing focusing by utilizing contrast.

According to an aspect of the embodiments, an ophthalmologic apparatus includes an illumination projection unit configured to project an illumination light onto a fundus of a subject's eye, an index projection unit configured to project an index onto the subject's eye, an image capturing unit configured to capture a fundus image including an index image being an image of the index based on light returning from the subject's eye, a focusing unit configured to focus the light returning from the subject's eye onto the image capturing unit, a detection unit configured to detect a position of the index image in the fundus image, a determination unit configured to determine an area in the fundus image which does not include the index image based on results detected by the detection unit, and a control unit configured to control the focusing unit based on contrast of the area determined by the determination unit.

According to another aspect of the embodiments, an ophthalmologic control method includes projecting an illumination light onto a fundus of a subject's eye, projecting an index onto the subject's eye, capturing a fundus image including an index image being an image of the index based on light returning from the subject's eye by an image capturing unit, detecting a position of the index image in the fundus image, determining an area in the fundus image which does not include the index image based on detected results, and controlling a focusing unit based on contrast of the determined area.

An ophthalmologic control program stored in a non-transitory computer readable medium is also disclosed.

Further features and aspects of the various embodiments will become apparent from the following detailed description with reference to the attached drawings.

### BRIEF DESCRIPTION OF THE DRAWINGS

The accompanying drawings, which are incorporated in and constitute a part of the specification, illustrate exemplary embodiments, features, and aspects of the application and, together with the description, serve to explain the principles of the invention.

FIG. 1A illustrates an exemplary embodiment of a fundus camera. FIG. 1B illustrates an example of a contrast AF detection area.

FIG. 2 illustrates a focus state detection unit according to the exemplary embodiment.

FIG. 3 is a flow chart illustrating an exemplary contrast AF process of the fundus camera.

FIG. 4 is a graph illustrating an exemplary function used in contrast AF detection.

FIG. 5 is an exemplary fundus image displayed on a display monitor.

### DETAILED DESCRIPTION

Various exemplary embodiments, features, and relevant aspects will be described in detail with reference to the drawings.

The techniques discussed below are not limited to the following exemplary embodiments, but may be modified and embodied in various forms without departing the general scope of the present exemplary embodiment.

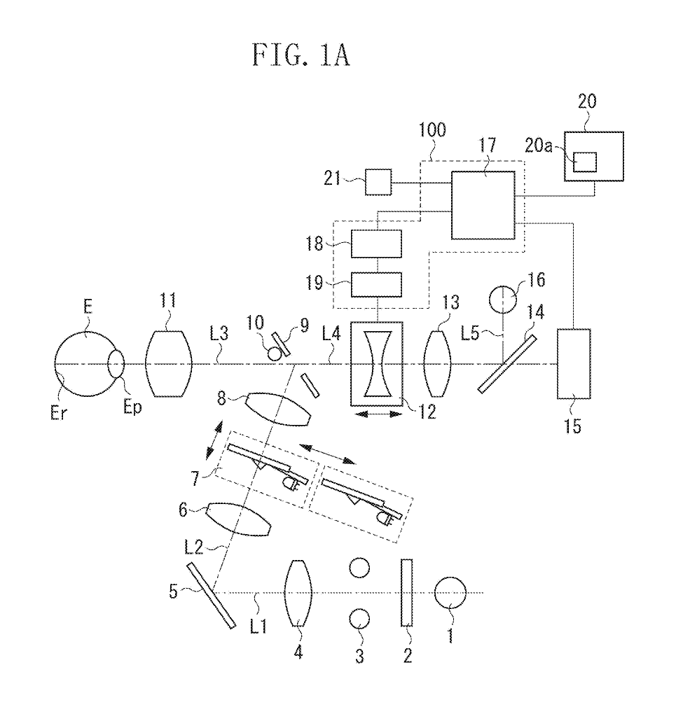

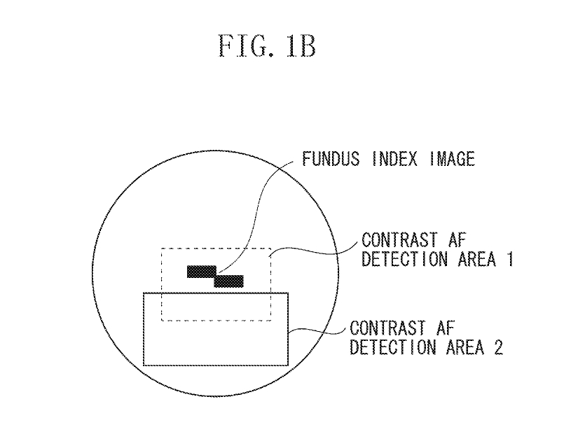

FIG. 1A is an example illustrating a general configuration of a fundus camera according to an exemplary embodiment. An illumination optical system is configured on optical axes L1 and L2. There are arranged an observation light source 1 emitting stationary light such as a halogen lamp, a filter 2 which transmits infrared light and shields visible light, a photographic light source 3 which emits visible light such as a flash unit, a relay lens 4, and a mirror 5 on the optical axis L1. There are sequentially arranged a relay lens 6, a focus index projection unit 7, a relay lens 8, and a perforated mirror 9 provided with a central aperture, on the optical axis L2 in the direction in which the mirror 5 reflects light.

The focus index projection unit 7 with the focus index is used for a coarse focus adjustment. The contrast of a fundus image described below is used for a fine focus adjustment. In other words, in the present exemplary embodiment, the coarse adjustment is performed using the focus index and then the fine adjustment is performed using the contrast of the fundus image to perform focusing. The focus index projection unit 7 uses the focus index in which an index is upward and downward split into two images in an out-of-focus state, as illustrated in FIG. 1B, for example, and two indexes are not split upward and downward in a focus state.

There are arranged an alignment-index light source 10 and an objective lens 11 opposing the subject's eye E on an optical axis L3 in the direction in which a perforated mirror 9 reflects light. On the rear side of the perforated mirror 9 are arranged a focus lens 12 which moves along an optical axis L4 to adjust focus, a photographic lens 13, a half mirror 14, and a fundus photographic unit 15 arranged on a predetermined surface conjugated with the fundus Er of the subject's eye E, as an observation photographic optical system. A fixation target display unit 16 formed of a light emitting diode (LED), for example, is arranged on an optical axis L5 in the direction in which the half mirror 14 reflects light.

The fundus photographic unit 15 is connected to a focus state detection unit 17. The focus state detection unit 17 is connected to a focus driving unit 19 for driving a focus lens 12 via a control unit 18. The focus state detection unit 17, the control unit 18, and the focus driving unit 19 form a driving control unit 100. The focus state detection unit 17 is connected to a display monitor 20 and a photographic switch 21. A focus detection area display portion 20a for displaying a focus detection area by a frame, for example, is provided on the screen of the display monitor 20.

The apparatuses other than the display monitor 20 and the photographic switch 21 are mounted on an optical base (not illustrated) to form an optical unit of the fundus camera. The optical unit is laid on a stage unit. The display monitor 20 may be integrated with the optical unit laid on the stage unit.

When the fundus camera is used, only infrared light among light flux emitted from the observation light source 1 passes through the filter 2 and passes through the photographic light source 3 such as a flash unit. The observation light source 1 is configured to function as an illumination projection unit for projecting illumination light onto the fundus of the subject's eye. The infrared light is concentrated by the relay lens 4, deflected by the mirror 5 in the direction of the optical axis L2, passes through the relay lenses 6 and 8, deflected again by the perforated mirror 9 in the direction of the optical axis L3, and illuminates the fundus Er of the subject's eye E via the objective lens 11.

The light flux reaching the fundus Er is reflected, scattered, and emitted from the subject's eye E as a fundus reflection image. The light flux passes through the objective lens 11, the optical axis L3, and the photographic lens 13 and forms an image on the fundus photographic unit 15. The focus state detection unit 17 controls to display the fundus image captured by the fundus photographic unit 15 on the display monitor 20.

An examiner roughly aligns the subject's eye E with the optical unit based on the alignment index image displayed on the display monitor 20 and then performs fine adjustment while observing the fundus image displayed on the display monitor 20.

Then, focus adjustment is performed. The photographic switch 21 is pressed to capture the fundus Er of the subject's eye. The focus adjustment is performed by the focus state detection unit 17 driving the focus lens 12 via the focus driving unit 19. The fundus camera according to the present exemplary embodiment has an automatic focusing (AF) function, which automatically executes the focus adjustment.

The light flux emitted from the fixation target display unit 16 is reflected by the half mirror 14 and reaches the subject's eye E via the photographic lens 13, the focus lens 12, the center aperture of the perforated mirror 9, and the objective lens 11. The examiner causes the subject to gaze a bright spot formed by the fixation target display unit 16 to regulate the subject's line of sight. The position of the bright spot of the fixation target display unit 16 is moved by a fixation target movement unit (not illustrated) to turn the subject's line of sight in any direction.

In a thus configured fundus camera, a focus is detected by detecting the contrast of the fundus image itself formed by a photographic light flux. This can solve a problem of the out-of-focus of the subject's eye due to lens aberration, which is the problem of a conventional apparatus using the focus index projected on an anterior eye Ep.

However, if the index image other than the fundus image is captured in the focus detection area (a contrast AF detection area) when the automatic focusing is performed by detecting the contrast, a contrast detection accuracy is reduced. The index image other than the fundus image includes a subject's eye aligning index (alignment index), a focusing detection index (focus index), and the fixation target, for example. As for the index light flux for aligning the subject's eye (alignment index), a parallel light flux with which a cornea is irradiated, for example, is reflected by the cornea to be used for alignment. On the other hand, a component passing through the cornea forms an image onto the fundus, which becomes a sharp index image when the fundus is observed. FIG. 1B illustrates an example of a case where an index image is included in the contrast AF detection area (a detection area 1) and a case where the index image is not included in the contrast AF detection area (a detection area 2).

Because the detection area 1 includes the fundus index image (the focus index image, for example), the detection accuracy of the contrast AF acquired by a calculation unit for calculating a contrast targeting the fundus image is reduced. On the other hand, the detection area 2 does not include the fundus index image (the focus index image, for example), so that it is possible to prevent reduction of the detection accuracy of the contrast AF targeting the fundus image. The detection area 2 is larger than the detection area 1 in FIG. 1B, however, which is not limited thereto. The size of the detection area may be adjusted to any size.

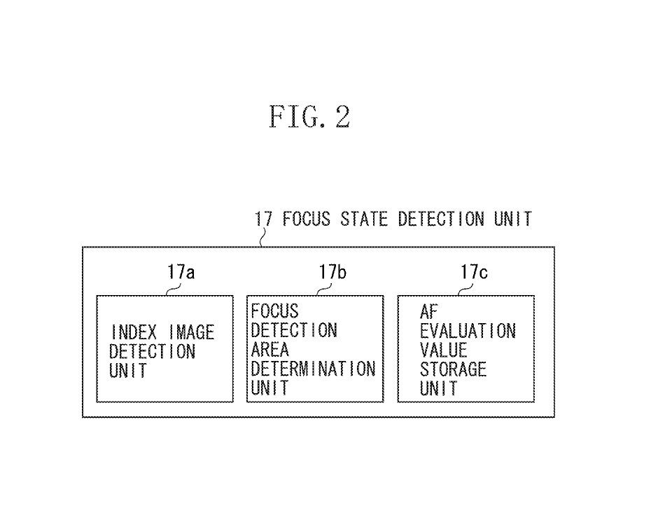

FIG. 2 is an example illustrating a configuration of the focus state detection unit 17 according to the present exemplary embodiment. As illustrated in FIG. 2, the focus state detection unit 17 incorporates an index image detection unit 17a for detecting the position of the index image of the index projected on the subject's eye. The focus state detection unit 17 detects a position as an index position where the output at each line of the horizontal direction of the fundus photographic unit 15 exceeds a reference level.

The focus state detection unit 17 further incorporates a focus detection area determination unit 17b for determining a focus detection area such that the index image is not included, based on the output of the index image detection unit 17a.

The focus state detection unit 17 still further incorporates an AF evaluation value storage unit 17c for storing an AF evaluation value and a position of the focus lens 12 at the time of calculating the evaluation value. The AF evaluation value storage unit 17c is a memory, for example.

Information about the position of the focus lens 12 is output from the control unit 18 to the focus state detection unit 17. In other words, the control unit 18 grasps the position of the focus lens 12.

[Flow Chart]

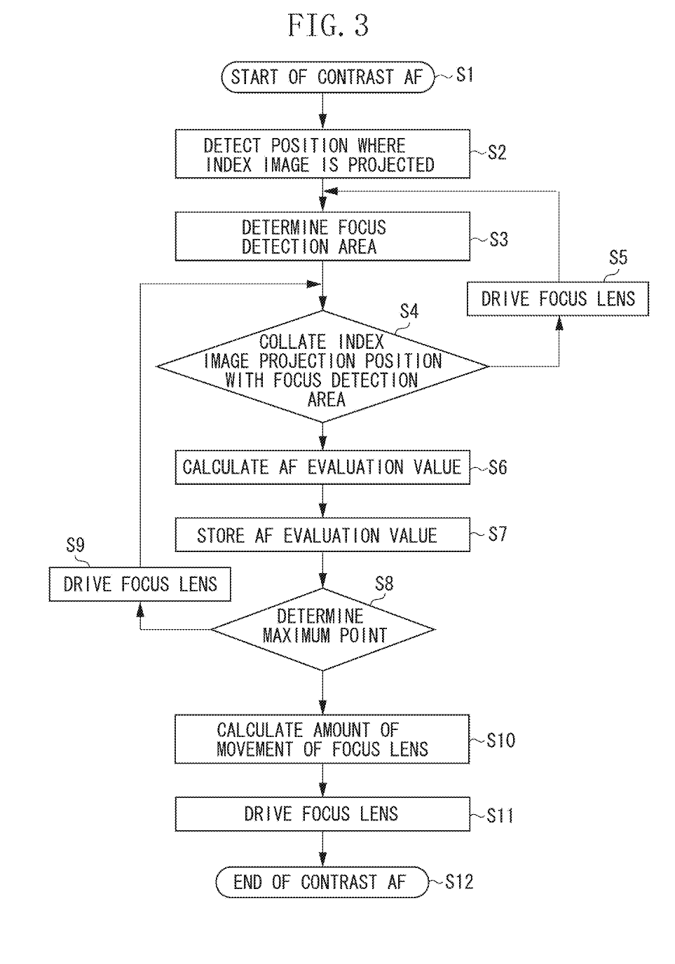

FIG. 3 is a flow chart illustrating an example of processing using the ophthalmologic apparatus according to the present exemplary embodiment. In step S1, an AF start switch (not illustrated) issues instructions for start of the contrast AF. The processing proceeds to step S2. In step S2, the index image detection unit 17a detects a projection position of the index image other than the fundus image from among the fundus image display area captured by the fundus photographic unit 15. In step S3, the focus detection area determination unit 17b sets a plurality of area candidates for performing the focus detection in the fundus image display area based on results detected by the index image detection unit 17a and determines one of the plurality of area candidates as the focus detection area.

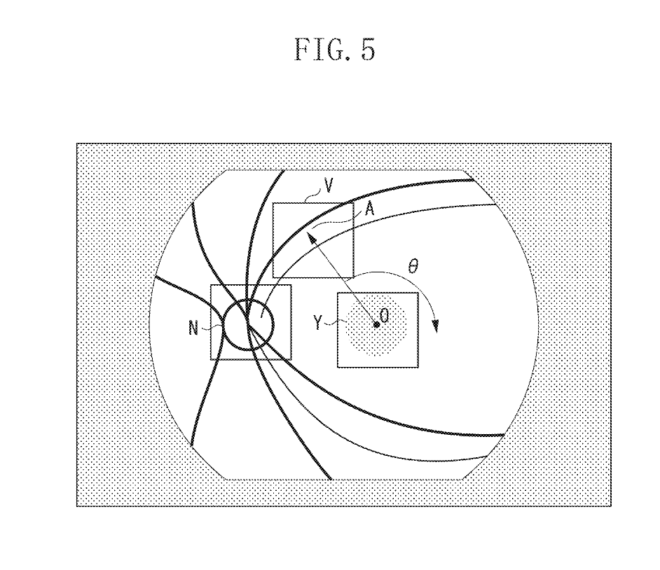

In step S2, if the focus index image (in FIG. 1B) exists in a yellow spot portion Y illustrated in FIG. 5, in step S3, a candidate of the focus detection area can be set in any direction to a yellow spot Y and with any size such that it does not include the index image. As illustrated in FIG. 5 which indicates the fundus image displayed on the display monitor, a middle blood vessel portion V can be set as a candidate of the focus detection area around a position A of viewing angle of 15 degrees as a center, in the direction of angle θ from a center O of the yellow spot portion Y. A nipple portion N illustrated in FIG. 5 can be set as a candidate of the focus detection area.

If there is a plurality of the focus detection areas, any candidate can be determined as a final focus detection area, so that a particular priority does not exist.

In step S4, the focus state detection unit 17 collates the index image projection position detected in step S2 with the focus detection area to determine whether the index image is included in the focus detection area. In step S4, the position of the index image in the fundus image displayed on the monitor) FIG. 5) is detected by reading a video signal of a scanning line passing through the index image

Determination is made as to whether the index image is included in the focus detection area in step S4 although the focus detection area has been determined such that the index image is not included, in step S3. The reason for that is the index image may be included in the focus detection area because the subject's eye is moved.

In step S4, if one index image or even a part thereof is included in the focus detection area, the processing proceeds to step S5. In step S5, one of the candidates of the focus detection area determined in step S3 is discarded. In step S5, it is assumed that the focus detection area is set again to make it a different focus detection area. For this reason, if the focus lens has been moved from the position taken when the AF is started, the control unit 18 transmits a signal to the focus driving unit 19 to return the focus lens to the position where the AF is started. The processing returns to step S3 and the next candidate is selected from among a plurality of the candidates previously set.

This cycle is repeated until a candidate of the focus detection area which does not include the index image is found. If the cycle is repeated the predetermined number of times, but a candidate is not found, this case can be treated as an error. In step S4, if the focus detection area which does not include the index image is found, the processing proceeds to step S6.

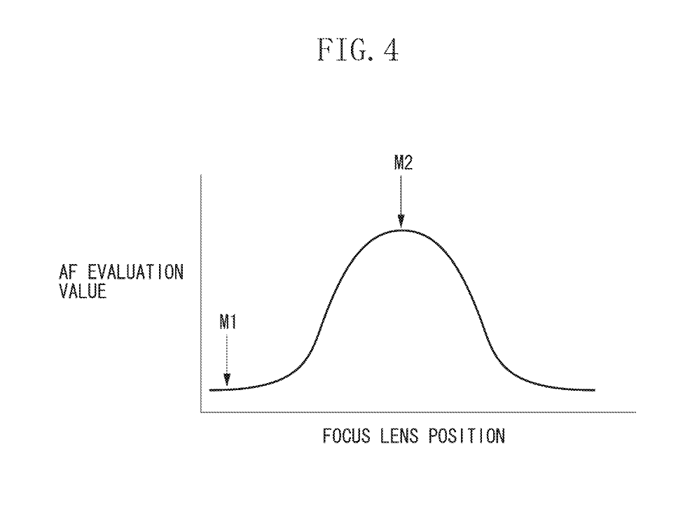

In step S6, the focus state detection unit 17 calculates the AF evaluation value of the focus detection area determined as including no index image in step S4, as a sensor output. A method for calculating the AF evaluation value is described below with reference to FIG. 4. In step S7, the AF evaluation value calculated in step S6 is stored in the AF evaluation value storage unit 17c by the focus state detection unit 17. In other words, the AF evaluation value storage unit 17c stores the AF evaluation value calculated in step S6.

As illustrated in steps S8 and S9, the focus lens is driven by increments until a maximum point of the AF evaluation value appears, and a change in the calculated sensor output is stored. A method for driving the focus lens by increments is such that, if the lens is driven while being staggeringly swung backward and forward in the optical axis direction with a constant amplitude, when the focus lens is away from the maximum point during the drive, the direction of the focus lens can be changed to the direction approaching the maximum point and the focus lens can quickly reach the maximum point. Another method for driving the focus lens is such that the focus lens may be driven by increments in a fixed direction without being swung.

FIG. 4 is a chart illustrating the principle of focus detection based on contrast detection. The focus detection method uses a specific high frequency component of the luminance signal which is maximized in an in-focus state. The focus state detection unit 17 detects a high frequency component of the input luminance signal and uses the high frequency component as the AF evaluation value. In FIG. 4, the abscissa represents the position of the focus lens 12 and the ordinate represents the amount of the AF evaluation value. The AF evaluation value is maximized at an in-focus position M2 and minimized at an out-of-focus position M1, for example. In the present exemplary embodiment, the principle of focus detection is used to correct focus adapted to observation aberration of a human eye optical system.

In step S8, the focus state detection unit 17 determines whether the maximum point that is the position M2 illustrated in FIG. 4 is included in the AF evaluation value stored in step S7, by using the principle of focus detection. At this point, in a first step S8, the focus state detection unit 17 cannot determine whether the maximum point is included therein, so that the processing proceeds to step S9. The focus driving unit 19 drives the focus lens 12 by a predetermined amount. The amount of driving the focus lens 12 may be changed by an inspector.

Again in step S4, the focus state detection unit 17 collates the index image projection position with the focus detection area each time contrast is calculated to determine whether the index image is included in the focus detection area. The reason the processing returns to step S4 is that the subject's eye may be moved such that the index image is included in the focus detection area. If the index image is included in the focus detection area, a new focus detection area is set via step S5.

In the above exemplary embodiment, the processing returns again to step S4 following step S9, but may return to step S6 without being limited to the above. This may lower the reliability of the AF evaluation value, but a high speed focusing is enabled. If the maximum point is not detected in the AF evaluation value, step S4 and steps S6 to S9 are repeated. If the determination process as to the maximum point has been repeated a predetermined number of times but the maximum point cannot be detected in the AF evaluation value, the process may be determined as an error by the focus state detection unit 17. In this case, the focus state detection unit 17 displays a message that an error has occurred, on the display monitor 20.

Alternatively, if the focus state detection unit 17 makes an error determination, focusing may be performed irrespective of whether the index image is included in the focus detection area. In this case, a message indicating that the index image can be included in the focus detection area may be displayed on the display monitor 20. Displaying such a message allows the inspector to easily grasp how focusing is performed and determine whether focusing is to be performed again.

In step S8, if the maximum point is detected in the AF evaluation value, the processing proceeds to step S10. The focus state detection unit 17 calculates the amount of movement of the focus lens 12. The amount of movement of the focus lens 12 refers to a travel distance of the focus lens 12 driven to a position where the maximum point of the AF evaluation value is detected.

Because the amount of the focus lens 12 to be driven to a position where the maximum point of the AF evaluation value is detected can be obtained using various known methods, a detailed description is omitted. In step S11, the control unit 18 transmits a signal to the focus driving unit 19 and the focus lens 12 is driven based on the amount of movement of the focus lens 12 calculated in step S8, and automatic focusing (contrast AF) is ended.

As described above, according to the present exemplary embodiment, the focus detection area which does not include the projection index image is selected and the contrast of the fundus image itself is detected, so that accurate focusing can be performed using the contrast evaluation without attenuating the light source for index of the fixation target. In other words, according to the present exemplary embodiment, the accuracy of automatic focusing can be improved. Furthermore, according to the present exemplary embodiment, a determination is made as to whether the index image is included in the focus detection area each time the focus lens is driven, so that a sure focusing can be performed based on the image of a fundus tissue.

[Modification 1]

In the above exemplary embodiment, a plurality of candidates for a focus detection area is previously set and one of them is selected and determined as the focus detection area. Alternatively, at least one of the position and the size of the focus detection area may be changed without previously setting candidates for the focus detection area if the index image exists in the focus detection area.

As far as the change of the focus detection area is concerned, the center position of the focus detection area may be changed without changing the size thereof, or the size of the focus detection area may be changed without changing the center position thereof. Alternatively, both the center position and the size of the focus detection area may be changed.

[Modification 2]

In the above exemplary embodiment, the fundus camera having only a function to capture the fundus of the subject's eye is described, but there may be an ophthalmologic apparatus which further includes a function to calculate a blood flow rate by capturing the fundus image of the subject's eye and measuring blood flow velocity and vascular caliber, for example. In other words, the fundus camera is an example of an ophthalmologic apparatus.

[Control of an Ophthalmologic Apparatus]

An ophthalmologic control method according to the embodiments disclosed herein includes projecting an illumination light onto the fundus of the subject's eye, projecting the index onto the subject's eye, and capturing the fundus image including the index image based on light retuning from the subject's eye. The ophthalmologic control method further includes detecting the position of the index image in the fundus image, determining the area which does not include the index image in the fundus image based on the detected results, and controlling a focusing unit based on the contrast of the determined area.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, these embodiments are not limiting. The scope of the following claims is to be accorded the broadest interpretation so as to encompass all such modifications and equivalent structures and functions.

This application claims the benefit of Japanese Patent Application No. 2012-236458 filed Oct. 26, 2012, which is hereby incorporated by reference herein in its entirety.

What is claimed is:

1. An ophthalmologic apparatus comprising:
an illumination projection unit configured to project an illumination light onto a fundus of a subject's eye;
an index projection unit configured to project an index onto the subject's eye;
an image capturing unit configured to capture a fundus image including an index image being an image of the index based on light returning from the subject's eye;
a focusing unit configured to focus the light returning from the subject's eye onto the image capturing unit;
a detection unit configured to detect a position of the index image in the fundus image;
a determination unit configured to determine an area of the fundus image which does not include the index image, based on results detected by the detection unit; and
a control unit configured to control the focusing unit based on contrast of the area determined by the determination unit.

2. The ophthalmologic apparatus according to claim 1, wherein the determination unit repeats determination of the area in the fundus image which does not include the index image if a state of the area is changed to that where the index image is included in the area.

3. The ophthalmologic apparatus according to claim 2, wherein the determination unit changes at least one of the size and the position of the area so that the index image is not included in the area if a state of the area is changed to that where the index image is included in the area.

4. The ophthalmologic apparatus according to claim 1, wherein:
the determination unit determines a plurality of areas in the fundus image which do not include the index image, and
the control unit controls the focusing unit based on the contrast of the other area among the plurality of areas if a state of the area is changed to that where the index image is included in one area used for controlling the focusing unit among the plurality of areas.

5. The ophthalmologic apparatus according to claim 2, further comprising:
a calculation unit configured to calculate contrast of the area; and
a determination unit configured to determine whether a state of the area is changed to that where the index image is included in the area each time the contrast is calculated by the calculation unit.

6. The ophthalmologic apparatus according to claim 1, wherein the index refers to at least one of a subject's eye aligning index, a focusing detection index, and a fixation target.

7. An ophthalmologic control method comprising:
projecting an illumination light onto a fundus of a subject's eye;
projecting an index onto the subject's eye;
capturing a fundus image including an index image being an image of the index based on light returning from the subject's eye by an image capturing unit;
detecting the position of the index image in the fundus image;
determining an area in the fundus image which does not include the index image based on detected results of the detecting step; and
controlling a focusing unit based on contrast of the determined area.

8. The control method of the ophthalmologic apparatus according to claim 7, wherein newly determining the area which does not include the index image in the fundus image if a state of the area is changed to that where the index image is included in the area.

9. The control method of the ophthalmologic apparatus according to claim 8, wherein changing at least one of the size and the position of the area so that the index image is not included in the area if a state of the area is changed to that where the index image is included in the area.

10. The control method of the ophthalmologic apparatus according to claim 7, wherein determining a plurality of areas which don't include the index image in the fundus image and controlling the focusing based on the contrast of the other area among the plurality of areas if a state of one area used for controlling the focusing among the plurality of areas is changed to that where the index image is included in the area.

11. A non-transitory storage medium recording an ophthalmologic control program, the program including coded instructions for causing a computer to execute the method according to claim 7.
